(19) United States
(12) Patent Application Publication
(10) Pub. No.: US 2011/0223224 A1
(43) Pub. Date: Sep. 15, 2011

(54) COSMETIC COMPOSITION INCLUDING MICROBEADS

(75) Inventors: Jean-Pierre Mathonnet, Le Cannet (FR); Gaetano Zannini, Biot (FR)

(73) Assignee: LABORATOIRES DOCTEUR GAETANO ZANNINI, Saint Raphael (FR)

(21) Appl. No.: 13/062,102

(22) PCT Filed: Sep. 3, 2009
(86) PCT No.: PCT/FR2009/001056

§ 371 Date: May 26, 2011

(30) Foreign Application Priority Data
Sep. 3, 2008 (FR) 0804832

(51) Int. Cl. A61K 8/02; A61K 8/06; A61Q 1/02; A61Q 1/10; A61Q 1/08; B05D 7/00; B05D 1/02; B05D 1/38; B05D 3/00; B05D 5/06

(52) U.S. Cl. 424/401; 424/63; 427/212; 427/214

(57) ABSTRACT

A cosmetic composition, microbeads to be added into a cosmetic carrier for said composition, methods for making the same, and the uses thereof. The composition comprises microbeads having one or more concentric coating layers around a core, each concentric coating layer including a solid phase and a binder, at least one of the concentric coating layers includes a pigment, and the composition also comprises a cosmetic carrier. The invention is particularly useful as a make-up product.

The present invention relates to a cosmetic composition, to microbeads to be added into a cosmetic carrier for said composition, to methods for making same and to the uses thereof, in particular as a make-up product.

The cosmetic compositions of the prior art are usually in solid form, e.g., in powdered form, or else in liquid form. If same are in liquid form, then they generally comprise suspensions or water-in-oil or oil-in-water emulsions of varying consistencies. The cosmetic compositions are most often colored according to the desired use. This is the case, in particular, with eye shadow or blush.

The color of the aforesaid cosmetic compositions of the prior art is identical to the color of the make-up resulting from the application of said compositions to the skin or skin appendages of the user, in particular. Thus, a red blush will remain red or a shade of this red color, once applied to the cheeks of the user. Admittedly, patent document FR 2 841 155 discloses a cosmetic product in the form of beads consisting of aggregates of grains agglomerated together by a binder. The grains are colored by means of pigments. The color of the outer layer of the aggregates is likely to differ from that of the inner layers. For use as a cosmetic product, the aggregates are dissociated and the grains comprising said aggregates are crushed so as to form a colored powder the final color of which, after the grains have been crushed, differs from that of the aggregates prior to crushing.

However, the cosmetic products disclosed in this patent document do not comprise any cosmetic carrier and the user then handles, not a conventional composition of the prior art, but only aggregates of grains, that must be dissociated by same prior to crushing the grains in order to obtain a powder.

In consideration of the above, one technical problem that the invention proposes to solve is that of providing an improved cosmetic composition, which optionally enables a make-up color to be selected which is separate from the color of the cosmetic product, and which, upon use, does not require dissociation of grain aggregates.

The first aim of the solution to this problem, as proposed by the invention, is a cosmetic composition, characterized in that same comprises microbeads having one or more concentric coating layers around a core, each concentric coating layer including a solid phase and a binder, and in that at least one of said concentric coating layers includes a pigment and in that the composition further comprises a cosmetic carrier.

The incorporation of pigments into concentric coating layers prevents said pigments from being released prematurely. In this way, during packaging, transport and storage, the pigments of the composition are not released into the cosmetic carrier. On the contrary, the pigments are released only at the moment when the composition is used, when the microbeads are crushed. Accordingly, in a composition which includes a carrier and a composition in microbead form according to the invention, the microbeads do not color the carrier before the composition is used as intended. In particular, the microbeads according to the invention have the advantage of not dissolving in oil or water, even under agitation, and of not being crushed in a cosmetic composition, in particular a cosmetic cream at rest.

The second aim of the invention is a microbead intended to be incorporated into a cosmetic carrier, characterized in that said microbead includes one or more concentric coating layers around a core, each concentric coating layer including a solid phase and a binder, in that at least one of said concentric coating layers includes a pigment, and in that the microbead further includes an outer shell.

The third aim of the invention is a method for manufacturing microbeads, characterized in that it includes the following steps:
granulation in the presence of a powdery solid phase and a liquid phase including a binder;
on or more consecutive spray coatings of a liquid phase including a binder, and dusting with a powdery solid phase; and
drying of the resulting microbeads upon completion of each coating step;

the powdery phase and/or the liquid phase, at the granulation step and/or at the coating step further including a pigment.

The fourth aim of the invention is the use of a composition according to the invention as a make-up product, preferably as an eye shadow, blush or make-up foundation.

Advantageously, within the scope of the present invention,—the microbeads further have an outer shell the composition of which is different from the composition of the coating layers;—the polarity of the outer shell is different from that of the cosmetic vehicle; the coating layers have a different polarity from that of the outer shell; the coating layers have a polarity identical or similar to that of the cosmetic carrier; the outer shell is hydrophobic when the cosmetic carrier is aqueous, or the outer shell is hydrophilic when the cosmetic carrier is lipidic or the outer shell is multilayered when the cosmetic carrier is both aqueous and lipidic;—the cosmetic composition is in the form of a powder, paste, cream, gel, lotion, water-in-oil or oil-in-water emulsion, suspension or solution;—the outer shell is hydrophilic or hydrophobic and/or multilayered; the method for manufacturing microbeads further includes a step for forming an outer shell by spraying a liquid or pasty phase and a solid phase onto the coated microbeads, the shell-forming step taking place after a final step for drying the coated microbeads;—the liquid or pasty phase includes a binding solution of animal waxes or Capol 600® in an alcohol, the alcohol having a strength greater than or equal to 70% alcohol by volume of alcohol of the total volume, in order to form a hydrophobic outer shell;—the liquid or pasty phase includes a binding solution of plant gum in an alcohol, the alcohol being diluted in water at a strength greater than or equal to 55% alcohol by volume of the total volume, in order to form a hydrophilic outer shell.

The invention will be better understood upon reading the following non-limiting disclosure.

The cosmetic composition according to the invention includes microbeads, also called granules. Each microbead includes a center portion forming the core, and a peripheral portion formed by one or more concentric coating layers around the core. Each of the concentric coating layers includes a solid phase and a binder. At least one coating layer includes at least one pigment.

According to one embodiment of the invention, the binder, solid phase and pigment composition is homogeneous from one coating layer to another, within each microbead. This enables microbeads to be obtained which release a constant color throughout the entire use thereof.

However, according to one particular embodiment of the invention, the composition can vary, and in particular the quantity and/or the nature of the pigment(s) can vary, from one coating layer of the microbead to another. This latter embodiment enables microbeads to be obtained which release a given pigment at a certain degree of wear, either from the first use or after a certain period of use of the microbeads. It also enables microbeads to be obtained which release various pigments during the use thereof and as the coating layers are being eroded. As a matter of fact, when the microbeads are used, for example, as a solid make-up product, the friction of the fingers or make-up brush erodes the coating layers one after the other, thereby gradually releasing the pigment or pigments contained therein.

The solid phase can be selected so as to consist of a cosmetic powder, such as talc or any powder for cosmetic use, a powder of dry pigments or else a cosmetic powder onto which pigments have been previously adsorbed, alone or as a mixture. The solid phase is preferably in loose powder form prior to formation of the microbead, and in compressed powder form in the formed microbead.

The pigment, which can be in the form of a pigment powder prior to formation of the microbead, is advantageously selected from dry powdered pigments which have simply been granulated, or else a grinding of pigments in an ester (isononyl isononanoate), in which the pigments have been adsorbed on a substrate and then granulated.

In the compositions in microbead form according to the invention, the pigment is present at a concentration of between 0.001 and 10% by weight, in relation to the total weight of the microbeads including same, preferably between 0.01 and 5% by weight, and in particular 0.75%, 1%, 1.5%, 2 or 3% by weight of the total weight of the microbeads.

The binder is advantageously selected from plant waxes and animal waxes, alone or as a mixture. The binder is advantageously used at the start in the form of a binding solution including a plant and/or animal wax diluted in alcohol, the alcohol being evaporated in the microbead ultimately obtained. A natural plant wax is preferred, in particular a carnauba wax and/or beeswax. A preferred animal wax is shellac. The mixture of carnauba wax, beeswax and shellac yields advantageous results. These binders enable the obtainment of microbeads having a degree of friability suitable for use of the composition as a cosmetic, in particular, and particularly as a make-up product. The microbeads according to the invention preferably have a friability of between 0.05 and 0.5 Newtons. The binder establishes an unstable bond between the grains of powder, whereby the microbeads crumble completely when crushed with the fingers. Thus, for example, in a blush or eye shadow in solid form, the preferred binder is a mixture of carnauba wax, beeswax and shellac and the microbeads have a preferred friability of between 0.05 to 0.5 Newtons.

In the microbeads according to the invention, the binder is present at a concentration of between 1 and 30% by weight, in relation to the total weight of the microbeads including same, preferably between 2 and 20%, and in particular between 4 and 15% by weight of the total weight of the microbeads.

Each microbead has a diameter of between 300 μm and 5 mm, preferably between 400 and 900 μm, preferably approximately 700 or 800 μm, shell excluded.

According to one advantageous embodiment, the microbeads further include an outer shell. The composition of the shell is different from that of the coating layers. It preferably includes a solid phase, or excipient, and a binder. The hardness of the shell is likely to be greater than that of the coating layers.

The solid phase used for the outer shell advantageously includes a powder selected from mica powder, Timiron® or talc, or any other powder for cosmetic use.

The solid phase can further include a pigment.

The shell of the microbeads can be of the same color as that of the coating layers. The shell can likewise be of a different color and, in this case, it enables the colored coating layers to be masked. Furthermore, if the microbeads are mixed with a cosmetic carrier, the outer shell can be of the color of said cosmetic carrier, e.g., of the same color as a cosmetic cream containing the microbeads. Or else, the shell can be of a different color from that of the cosmetic carrier. The shell can likewise be transparent. This shell enables the microbeads to be incorporated into a liquid composition, e.g., an aqueous or oily or mixed external phase. The shell does indeed enable the microbeads to protected, and in particular to be protected from premature degradation, by solubilization, irrespective of the carrier and/or the type of emulsion in which same are mixed.

The outer shell enables the microbeads to not become solubilized in the cosmetic composition upon contact with the cosmetic carrier with which same are mixed, and to not disintegrate before the composition is used as intended.

To accomplish this, the shell has an opposite polarity from that of the cosmetic carrier with which the microbeads are intended to be mixed. The shell is thus substantially polar when the cosmetic carrier is apolar, and vice versa. In particular, the shell is substantially hydrophobic when the cosmetic carrier is substantially aqueous, and has a hydrophilic character. Conversely, the shell is substantially hydrophilic when the cosmetic carrier is substantially lipidic, and has a hydrophobic character.

The coating layers can have a different polarity from that of the outer shell. In this case, when the shell disintegrates, into the form of a fine powder, e.g., e.g., by means of an applicator brush, the underlying coating layers are exposed. They are then in contact with the cosmetic carrier in which they are solubilized. They thereby gradually and homogeneously release the compounds contained therein, and in particular the pigments, into the carrier.

Conversely, the coating layers can have a polarity identical or similar to that of the outer shell. In this case, when the shell disintegrates, the underlying coating layers are not easily solubilized upon contact with the cosmetic carrier. For example, the compounds which same contain, and in particular the pigments, are only released by the mechanical action of a brush.

The binder and the powder used for the outer shell determine the polarity thereof, i.e., the hydrophilic or hydrophobic character thereof.

In order to obtain a hydrophobic outer shell, the preferred binder is selected from a binding solution of animal waxes, Capol 600® or any other solution for cosmetic use, in an alcohol. The alcohol is an absolute or high-strength alcohol, preferably greater than or equal to 70% alcohol by volume of alcohol of the total volume. After drying, the alcohol is no longer present except in trace amounts in the shell of the microbeads. The solution preferably includes from 5 to 50% binder by weight of the total weight of the binding solution.

When a few microbeads provided with a hydrophobic shell are added to water, the completely waterproof shell is resistant thereto. The microbeads preserve the initial appearance thereof over time (one-month test with thermal shocks). The microbeads were thus able to be preserved under normal laboratory conditions, under natural and artificial light, at a temperature of approximately 22° C., +/−4° C., under agitation, without any modification in the initial appearance thereof. On the other hand, when these same microbeads provided with a hydrophobic shell are poured into vegetable oil, the shell is solubilized in a few minutes, and the oil-soluble pigments solubilize in turn. The pigments are solubilized in the oil and the oil assumes the color of the pigment in a few seconds under agitation and in a few minutes when at rest.

In order to obtain a hydrophilic outer shell, the binder preferred is selected from a plant gum binding solution, like gum arabic, acacia gum or carob bean gum, or any other solution for cosmetic use, in an alcohol diluted in water. The alcohol dilution in water is preferably less than or equal to 55% alcohol, and preferably 30% by volume of the total volume of the dilution. After drying, the alcohol is no longer present except in trace amounts in the shell of the microbeads. Thus, the binder is in the form of a solution prior to formation of the shell, but more so when the outer shell is formed. The solution preferably includes from 5 to 50% binder by weight of the total weight of the binding solution.

When a few microbeads with a hydrophilic shell are poured into oil, the completely oil-proof shell is resistant thereto. The microbeads preserve the initial appearance thereof over time (testing over several months). The microbeads were thus able to be preserved under normal laboratory conditions, under natural and artificial light, at a temperature of approximately 22° C., +/−4° C., under agitation, without any modification in the initial appearance thereof. On the other hand, when these same microbeads provided with a hydrophilic shell are poured into pure water, or an aqueous solution, the shell is solubilized in a few minutes, and the pigments slightly soluble in the water phase are slightly solubilized or not at all in the water. Only the shell disappears in a few seconds under agitation and in a few minutes when at rest. The pigments preserve the granulated appearance thereof in the water, and the water assumes the appearance of a suspension with small suspended beads of pigments.

In order to obtain a hydrophilic outer shell, the Timiron® solid phase yields better results than the talc, in particular in combination with a 10% gum binding solution in alcohol, at 50° or else with a 1% hydroxypropylmethyl cellulose (HPMC) solution in alcohol, at 50°. As a matter of fact, in sweet almond oil, partial dissolution of the talc shell is noted but not the Timiron® shell.

The combinations of Timiron®, or of Timiron® and carob bean gum as a solid phase and a 10% gum solution in alcohol at 50° as a binder, are preferred, because the microbeads thus encased do not dissolve in sweet almond oil and because the shell formation stage is done without the formation of non-desirable blocks of granules. The combination of Timiron® and acacia gum as a solid phase and a 10% gum solution in alcohol at 50° as a binder, is also preferred, because the microbeads thus encased do not dissolve in sweet almond oil or in beauty lotion.

According to one advantageous embodiment, the outer shell is a multilayered shell. It enables the microbeads to be protected from solubilization when same are incorporated into a cosmetic carrier which is both aqueous and lipidic. As a matter of fact, it is common in cosmetics to use carriers which are not strictly aqueous or strictly oily, like water-in-oil (W/O) or oil-in-water (O/W) emulsions. A multilayered shell according to the invention is insoluble in water and in oil. A multilayered shell insoluble in an O/W emulsion preferably includes a hydrophilic shell covered with a hydrophobic shell, and a multilayered shell insoluble in a W/O emulsion preferably includes a hydrophobic shell covered with a hydrophilic shell. As a matter of fact, in a W/O emulsion, the microbeads are in contact substantially with the lipidic phase, the aqueous phase being present in the form of droplets surrounded by surfactants, whereas, in an O/W emulsion, the microbeads are in contact substantially with the aqueous phase, the lipidic phase being present in the form of droplets surrounded by surfactants. The applicant surprisingly observes improved stability of the microbeads with multilayered shells, in O/W or W/O emulsions, in comparison with microbeads with only a hydrophobic shell or a hydrophilic shell (depending on the circumstances).

The microbeads according to the invention thus have the following advantageous characteristics:
good granulometric consistency;
satisfactory pigment color and satisfactory shell color;
high microbead stability when mixed into a cosmetic carrier;
high microbead stability over time;
masking of the microbeads in a cosmetic carrier, in particular a cream;
good restitution of the microbead pigment color when applied to the skin;
in the case of a make-up foundation including microbeads according to the invention, the color of the make-up foundation can be modulated by adjusting the color of the pigment, the size of the pigmented microbeads, the color and thickness of the protective outer shell, and the proportion of pigmented microbeads in the cosmetic carrier.

The cosmetic composition according to the invention further includes a cosmetic carrier. Said cosmetic carrier can be solid or liquid, polar or apolar. It is likewise cosmetically acceptable, i.e., it is suitable for use in contact with human or animal cells, without being toxic, irritating, producing any undue allergic response or the like, and while being proportioned in a reasonable advantage/risk ratio. The microbeads are dispersed in the cosmetic carrier. In other words, they do not aggregate to form aggregates. The microbeads according to the invention are under no circumstances bound together by a binder with a view to forming aggregates. As a result of the above, the cosmetic composition according to the invention, comprising a cosmetic carrier, has a homogenous appearance.

The solid cosmetic carrier is advantageously a powder.

The liquid cosmetic carrier is advantageously selected from water, an oil, an alcohol, alone or in a mixture, an emulsion, suspension, gel, cream, lotion or solution, e.g., an aqueous, alcohol, hydroalcoholic or fatty solution. An emulsion can be of the water-in-oil (W/O) or oil-in-water (O/W) type and results from placing an oily phase in contact with an aqueous phase in the presence of surfactants. The direction of the emulsion (W/O or O/W) is determined by the mode of operation and nature of the oils and surfactants used. An O/W emulsion is more fluid and less fatty than a W/O emulsion.

The cosmetic composition according to the invention is intended for topical use.

The composition according to the invention can be in various forms acceptable for topical use, in particular in a solid form or liquid form, based on the carrier used. It can thus be in the form of a more or less granular powder, due to the presence of the microbeads, or in the form of a cream, paste, gel, lotion, emulsion or solution.

The cosmetic composition is advantageously a make-up product, preferably an eye shadow, blush or make-up foundation. The make-up product can be in solid form, preferably in powdered form, or in liquid form, preferably in the form of a cream.

The invention likewise relates to a method for manufacturing a composition in microbead form according to the invention.

The method according to the invention includes the following steps:
granulation in the presence of a powdery solid phase, which is preferably crystallized, and a liquid phase including a binder;
one or more consecutive spray coatings of a liquid phase including a binder, and dusting with a powdery solid phase, which is preferably crystallized; and
drying of the resulting microbeads upon completion of each coating step;

the powdery phase and/or the liquid phase, at the granulation step and/or at the coating step including a pigment.

This method is carried out in at least one turbine rotating about a spindle for the coating operation. The spindle of each rotating turbine is inclined at an angle of 45 to 60° relative to horizontal.

The method is carried out at a temperature of between 18 and 25° C., except for the drying operation, which is carried out at a maximum of 45° C. in an oven. It is carried out at less than 70% humidity and at a suction rate of 2,500 m³ per hour.

The first step is the granulation step. It enables formation of the center portion of the microbead, referred to as the core.

The granulation step is followed by one or more coating and drying steps.

Each coating step enables the formation of a coating layer around the core or a previous coating layer.

At the granulation step and at the coating step, the solid phase is powdery, and preferably crystallized. As described above, it advantageously includes a cosmetic powder, a dry pigment powder or else a cosmetic powder onto which pigments have been previously adsorbed.

The method according to the invention thus enables granulation of cosmetic powders, pigment powders and/or cosmetic powders onto which pigments have been previously adsorbed.

The liquid phase can include water and/or alcohol.

When the binder is included in the liquid phase, the latter constitutes a binding solution the density of which is greater than that of water. The binder used preferably has a density of between 1010 and 1020 and a melt index of between 16 and 20.

The granulation, coating, drying and sieving steps can be carried out as described and under the conditions stated in the French patent applications published as FR 2 737 134 and FR 2 816 518.

The size of the microbeads obtained by the method according to the invention is directly linked to the duration of the coating step and therefore to the number of concentric coating layers.

According to a preferred embodiment, the liquid phase/solid phase weight ratio used in each coating step is between 1/4 and 1/5.

The drying steps can be carried out in an oven, at a temperature of between 40 and 55° C., and preferably 45 to 50° C., for 1 hr. After each drying step, the alcohol and water are evaporated. This enables microbeads having dry pigments to be obtained.

The microbeads obtained by means of the aforesaid method therefore consist of a center portion forming the core, and a peripheral portion formed by one or more concentric coating layers that have been deposited consecutively according to the method.

According to one advantageous embodiment, the method according to the invention further includes a sieving step for obtaining calibrated microbeads, said sieving step taking place after a final step for drying the coated microbeads.

According to one advantageous embodiment, the method according to the invention further includes a step for forming an outer shell by spraying a liquid or pasty phase and a solid phase onto the coated microbeads, the shell-forming step taking place after a final step for drying the coated microbeads, before or after the calibrating step.

The binder used for the outer shell is introduced either with the solid phase or with the liquid phase. In liquid form, the binder is preferably a binding solution.

The binder and the solid phase used for the outer shell are as defined above.

The method according to the invention thus enables the microbeads obtained with an outer protective shell to be covered.

According to a preferred embodiment, the formation of a hydrophobic or hydrophilic outer shell first includes impregnating the microbeads with a binding solution or liquid phase, as described previously for a hydrophobic or hydrophilic outer shell, respectively, and preferably in a sugar-coating turbine. The microbeads become sticky.

Next, as described previously for the outer shell, the liquid phase is sprayed onto said microbeads in the sugar-coating turbine. Said solid phase is thus evenly distributed on the surface of the microbeads when the microbeads make contact with one another in the rotating turbine.

These impregnating and spraying steps can be repeated, generally twice, until a white, homogenous shell is obtained, or a shell of a different color from that of the microbeads.

Finally, formation of the shell includes drying the microbeads in the oven for approximately 1 hr, at a temperature of approximately 40° C. After this drying step, only residual traces of alcohol remain.

The formation of a multilayered outer shell is carried out by successively applying the appropriate aforesaid methods.

The invention likewise relates to the compositions obtained by the method according to the invention.

The invention likewise relates to the use of a composition according to the invention as a make-up product, and preferably as an eye shadow, blush or make-up foundation, in solid or liquid form.

Listed hereinbelow are examples of the ingredients and uses thereof for preparing compositions in microbead form according to the invention. The proportions of the ingredients are indicated in percentage by weight of the total composition. This involves exemplary starting compositions, because the resulting final compositions hardly include anymore alcohol or water, which evaporated during the drying steps:

### EXAMPLE 1

### Eye Shadow Microbeads “Shadow 715”

Shadow 715: 74.88%

95% alcohol: 10.05%

Natural waxes: 10.05%

(carnauba, beeswax, shellac)

Purified water: 5.02%

The binder (natural waxes) is hydrophobic and the microbeads have a good degree of stability in the O/W emulsions.

### EXAMPLE 2

### Eye Shadow Microbeads “Shadow 775”

Shadow 775: 75.07%

95% alcohol: 9.97%

Natural waxes: 9.97%

(carnauba, beeswax, shellac)

Purified water: 4.99%

The binder (natural waxes) is hydrophobic and the microbeads have a good degree of stability in the O/W emulsions.

### EXAMPLE 3

### Fluid Make-Up Foundation Microbeads

Microbead:
Crushed dry pigments 69.75%
95% alcohol 10.10%
Natural waxes 10.10%
(carnauba, beeswax, shellac)
Purified water 5.05%

Shell:
Timiron 4.00%
Gum arabic solution 1.00%

The binder (gum arabic solution) is hydrophilic and the microbeads have a good degree of stability in the W/O emulsions.

### EXAMPLE 4

### Make-Up Foundation Microbeads

Microbead:
Crushed dry pigments 69.75%
95% alcohol 10.10%
Natural waxes 10.10%
(carnauba, beeswax, shellac)
Purified water 5.05%

Shell:
Timiron+gum arabic 4.00%
Gum arabic solution 1.00%

The resulting shell is water-soluble. It has better stability in oils than the shell of the preceding example.

### EXAMPLE 5

### Make-Up Foundation Microbeads

Microbead:
Crushed dry pigments 69.75%
95% alcohol 10.10%
Natural waxes 10.10%
(carnauba, beeswax, shellac)
Purified water 5.05%

Shell:
Timiron+gum arabic 4.00%
Gum arabic solution 1.00%

The binder (gum arabic solution) is hydrophilic and the microbeads have a good degree of stability in the W/O emulsions.

### EXAMPLE 6

### Fluid Make-Up Foundation Microbeads

Microbead:
Crushed dry pigments 69.75%
95% alcohol 10.10%
Natural waxes 10.10%
(carnauba, beeswax, shellac)
Purified water 5.05%

Shell:
Timiron+gum arabic 4.00%
Gum arabic solution 0.50%
Natural waxes 0.50%
(carnauba, beeswax, shellac)

The resulting shell is water-soluble. It has improved stability in an oily medium, owing to the gum arabic solution, which produces a hydrophilic surface film on the shell.

### EXAMPLE 7

### Fluid Make-Up Foundation Microbeads

Microbead:
Powder: talc 66.17%
Liquid pigments: grinding of pigments in an ester (isononyl isononanoate) 15.57%
95% alcohol 9.51%
Natural waxes 3.75%
(carnauba, beeswax, shellac)

Shell:
Timiron 3.60%
Gum arabic 0.40%
Gum arabic solution 1.00%

The resulting shell is water-soluble. It has a good degree of stability in an oily medium.

The microbeads are then incorporated into a cream in the amount of 7% by weight of the total composition.

### EXAMPLE 8

### Fluid Make-Up Foundation Microbeads

Microbead:
Powder: talc 66.17%
Liquid pigments: grinding of pigments in an ester (isononyl isononanoate) 15.57%
95% alcohol 9.51%
Natural waxes 3.75%
(carnauba, beeswax, shellac)

Shell:
Timiron 3.60%
Gum arabic solution 0.50%
Natural waxes 0.50%
(carnauba, beeswax, shellac)

The resulting shell is water-soluble. It has a good degree of stability in an oily medium, owing to the gum arabic solution, which produces a hydrophilic surface film on the shell.

The microbeads are then incorporated into a cream in the amount of 7% by weight of the total composition.

### EXAMPLE 9

### Fluid Make-Up Foundation Nicrobeads

Microbead:
Powder: talc 69.83%
Liquid pigments: grinding of pigments in an ester (isononyl isononanoate) 15.10%
95% alcohol 10.07%

Shell:
Timiron 3.60%
Gum arabic solution 0.50%
Natural waxes 0.50%
(carnauba, beeswax, shellac)

The resulting shell is water-soluble. It has improved stability in an oily medium, owing to the gum arabic solution, which produces a hydrophilic surface film on the shell.

The microbeads are then incorporated into a cream in the amount of 7% by weight of the total composition.

### EXAMPLE 10

### Fluid Make-Up Foundation Microbeads

Microbead:
Powder: talc 69.83%
Liquid pigments: grinding of pigments in an ester (isononyl isononanoate) 15.57%
95% alcohol 10.07%

Shell:
Timiron 3.60%
Gum arabic 0.40%
Gum arabic solution 1.00%

The resulting shell is water-soluble. It has a good degree of stability in an oily medium.

The microbeads are then incorporated into a cream in the amount of 10% by weight of the total composition.

### EXAMPLE 11

### Example of Ingredients Used to Make a Hydrophilic Outer Shell

Solid phase: Timiron

Liquid phase: 10% gum solution in alcohol at 50°

### EXAMPLE 12

### Example of Ingredients Used to Make a Hydrophilic Outer Shell

Solid phase:
Timiron 90%
Acacia gum 10%

Liquid phase:
10% gum solution in alcohol at 50°

### EXAMPLE 13

### Example of Ingredients Used to Make a Hydrophilic Outer Shell

Solid phase:
Timiron 80%
Carob bean gum 20%

Liquid phase:
10% gum solution in alcohol at 50°

### EXAMPLE 14

### Example of Ingredients Used to Make a Hydrophilic Outer Shell

Solid phase:
Timiron 90%
Carob bean gum 10%

Liquid phase:
10% gum solution in alcohol at 50°

### EXAMPLE 15

### Example of Ingredients Used to Make a Multilayered Outer Shell

1^{st} Solid phase: Timiron

1^{st} Liquid phase: 10% gum solution in alcohol at 50°

2^{nd} Solid phase: Timiron

2^{nd} Liquid phase:
Capol solution 600® 60%
in alcohol at 96° 40%

The invention, of course, is not limited to the embodiments described above, and a person skilled in the art, using routine operations, would be able to adapt said embodiments.

1. A cosmetic composition comprising microbeads having one or more concentric coating layers around a core, each concentric coating layer including a solid phase and a binder, wherein at least one of said concentric coating layers includes a pigment and wherein the composition further comprises a cosmetic carrier.

2. The composition according to claim 1, wherein the microbeads further have an outer shell whose composition is different from the composition of the coating layers.

3. The composition according to claim 2, wherein the polarity of the outer shell is different from that of the cosmetic carrier.

4. The composition according to claim 2, wherein the coating layers have a different polarity from that of the outer shell.

5. The composition according to claim 1, wherein the coating layers have an identical or similar polarity to that of the cosmetic carrier.

6. The composition according to claim 2, wherein the outer shell is hydrophobic when the cosmetic carrier is aqueous, or the outer shell is hydrophilic when the cosmetic carrier is lipidic, or the outer shell is multilayered when the cosmetic carrier is both aqueous and lipidic.

7. A cosmetic composition according to claim 1, which is in the form of a powder, paste, cream, gel, lotion, water-in-oil or oil-in-water emulsion, suspension or solution.

8. A microbead intended to be incorporated into a cosmetic carrier, wherein said microbead includes one or more concentric coating layers around a core, each concentric coating layer including a solid phase and a binder, wherein at least one of said concentric coating layers includes a pigment, and wherein the microbead further includes an outer shell.

9. The microbead according to claim 8, wherein the outer shell is hydrophilic or hydrophobic and/or multilayered.

10. A method for manufacturing microbeads, including the following steps:
granulation in the presence of a powdery solid phase and a liquid phase including a binder;
on or more consecutive spray coatings of a liquid phase including a binder, and dusting with a powdery solid phase; and
drying of the resulting microbeads upon completion of each coating step;
the powdery phase and/or the liquid phase, at the granulation step and/or at the coating step further including a pigment.

11. The method according to claim 10, further including a step for forming an outer shell by spraying a liquid or pasty phase and a solid phase onto the coated microbeads, the shell-forming step taking place after a final step for drying the coated microbeads.

12. The method according to claim 11, wherein the liquid or pasty phase includes a binding solution of animal waxes or Capol 600® in an alcohol, the alcohol having a strength greater than or equal to 70% alcohol by volume of alcohol of the total volume, in order to form a hydrophobic outer shell.

13. The method according to claim 11, wherein the liquid or pasty phase includes a binding solution of plant gum in an alcohol, the alcohol being diluted in water at a strength greater than or equal to 55% alcohol by volume of the total volume, in order to form a hydrophilic outer shell.

14. A method of applying make-up to the skin, comprising applying a composition according to claim 1 as a make-up product.

15. The method of claim 14, which is a method of applying eye shadow.

16. The method of claim 14, which is a method of applying blush.

17. The method of claim 14, which is a method of applying make-up foundation.
